(19) United States
(12) Patent Application Publication
(10) Pub. No.: US 2015/0313982 A1
(43) Pub. Date: Nov. 5, 2015

(54) METHODS AND COMPOSITIONS OF PROTEIN ANTIGENS FOR THE DIAGNOSIS AND TREATMENT OF LEPTOSPIROSIS

(71) Applicants: THE REGENTS OF THE UNIVERSITY OF CALIFORNIA, Oakland, CA (US); IMMPORT THERAPEUTICS, INC., Irvine, CA (US); YALE UNIVERSITY, New Haven, NH (US)

(72) Inventors: Philip FELGNER, Rancho Santa Fe, CA (US); Xiaowu LIANG, La Jolla, CA (US); Albert KO, Irvine, CA (US); Elisio A. WUNDER, JR., New Haven, CT (US)

(21) Appl. No.: 14/651,960

(22) PCT Filed: Dec. 12, 2013
(86) PCT No.: PCT/US13/74663

§ 371 Date: Jun. 12, 2015

Related U.S. Application Data
(60) Provisional application No. 61/736,391, filed on Dec. 12, 2012.

(51) Int. Cl. A61K 39/02; G01N 33/569

(52) U.S. Cl. CPC A61K 39/0225; G01N 33/56911; G01N 2333/20; G01N 2469/20; G01N 2800/26

(57) ABSTRACT

Novel immunodominant antigenic proteins and peptides associated with associated with leptospirosis were identified using a proteome array based on expression of ORFs from a Leptospira genome. Compositions, methods, and uses of such antigenic proteins and peptides in the diagnosis and staging of leptospirosis infection and in compositions, methods, and uses of such antigenic is proteins and peptides in prophylactic and therapeutic vaccines are disclosed.

This application claims priority to Provisional Application No. 61/736,391 filed on Dec. 12, 2012. These and all other referenced extrinsic materials are incorporated herein by reference in their entirety. Where a definition or use of a term in a reference that is incorporated by reference is inconsistent or contrary to the definition of that term provided herein, the definition of that term provided herein is deemed to be controlling.

### FIELD OF THE INVENTION

The field of the invention is compositions and methods for diagnosis and treatment of various disorders and diseases, in particular leptospirosis.

### BACKGROUND

Leptospirosis is an important, widespread disease caused by infection with bacteria of the genus Leptospira. The disease is transmitted to humans from contact with infected domestic or wild animals, or by contact with their urine. A wide variety of animal species can act as reservoirs for the bacteria. As a result, human leptospirosis is often considered to be the most widespread zoonotic disease. Groups that are traditionally at high risk for leptospriosis include, farmers, veterinarians, members of the military, miners, and individuals that work with sewage. Alternative patterns of transmission, however, have also been observed. Outbreaks of leptospirosis have been associated with recreational activities, such as ecotourism and sporting events that emphasize outdoor activities.

Individuals with leptospirosis show a wide variety of relatively nonspecific clinical symptoms in the early stages of the illness, including fever, headache, chills, and severe muscle pain, making early diagnosis on the basis of clinical findings difficult. Unfortunately, 5-15% of infections result in severe multisystem complications, including jaundice, kidney failure, and hemorrhaging. Severe leptospirosis has a mortality rate of 5-40%. Conditions associated with severe poverty have led to epidemics of leptospirosis in urban areas of Brazil and other countries, with high mortality rates.

The lack of a rapid and reliable point-of-care diagnostic test is a major barrier not only to assessing the global burden of the disease but also to providing an early diagnosis. Current diagnostic tests primarily rely on detection of antibodies binding to leptospiral lipopolysaccharide (LPS) and are insensitive in early illness, when antibiotic therapy is most effective.

Diagnosis of leptospirosis is difficult, particularly in early, treatable stages of the disease. Currently the most direct demonstration of the presence of Leptospira is by either isolation and culture or PCR. Unfortunately, bacteriological isolation is an expensive process, requiring highly trained technicians and specialized facilities. In the case of leptospirosis this is further complicated by the sensitive nature of Leptospira, which requires the use of very fresh samples. In addition, the organism grows relatively slowly in culture, with results requiring several weeks to develop. PCR has therefore proven to be a more useful tool for identification of Leptospira in a sample. PCR methods, however, still require specialized equipment and a high level of skill to perform properly, and, while sensitive, may not be sufficiently quantitative to assess the stage of Leptospira infection.

Leptospirosis can also be diagnosed using serological methods, which characterize an individual's immunological response to the organism. The current reference method for serological diagnosis of leptospirosis is a microagglutination test (MAT), in which the ability of a patient's serum to agglutinate cultured Leptospira is characterized. Unfortunately, this method is limited by the need to culture Leptospira for use in the assay and the heterogeneity of the organism.

Attempts have been made to identify and produce specific Leptospira proteins or peptides that can be useful in traditional immunoassays (such as enzyme immunoassays or lateral flow tests) for the diagnosis of leptospirosis. For example, in European Patent No. 2205625B1 (to Chang) and United States Patent Application No. 2012/0100143 (to Chang) a number of Leptospira outer membrane proteins (i.e. LP 1454, LP 1118, LP 1939, MCEII, CADF-likel, CADF-like2, CADF-like3, Lp0022, Lp1499, Lp4337, Lp328, L21) and surface proteins (i.e. LigA, and LigB) are disclosed for use in the diagnosis and prevention of leptospirosis. Similarly, U.S. Pat. No. 7,531,177 (to Nascimento et al) describes the use of surface associated proteins LpL53, OMPL55, OMPL16, OMPL31, OMPL15, OMPL20, LpL23, LpL22, OMPL17, OMPL30, OMPL27, OMPL21, OMPL22, MPL17, MPL21, OMPAL21, OMPL63, OMPL14, MPL36, MPL39, MPL40, and MPL21 as antigens for use as vaccinating species and in immunological assays for Leptospira. The pre-selection of potential antigens in such approaches, however, limit them to Leptospira surface proteins which, while accessible by an individual's immune system, may not include useful targets.

Other investigators have used various methods to identify potential Leptospira antigens from a broader variety of proteins. For example, U.S. Pat. No. 7,635,480 (to Andre-Fontaine et al) discloses the use sera generated by immunization with whole cells from a pathogenic strain of Leptospira to label proteins from both pathogenic and non-pathogenic strains; subsequent differentiation between the proteins thus labeled resulted in the identification of a peptide useful in the prevention and identification of leptospirosis. In another approach, described in U.S. Pat. No. 8,445,658 (to Ko et al), a phage library of the Leptospira genome was developed and immune serum from convalescing leptospirosis patients was used to select clones with proteins that had elicited an immune response. Several previously unidentified protein antigens (i.e. BigL1, BigL2, and BigL3) were identified, along with a number of previously known Leptospira antigens. While such approaches reduce the bias introduced by pre-selection of specific classes of proteins, they simply identify proteins and/or protein fragments to which antibodies have been elicited. Such approaches fail, however, to specifically identify and provide Leptospira antigens that generate the particularly strong and/or widespread antibody responses that are useful for diagnostic and preventative purposes.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Thus, there is still a need for immunodominant Leptospira antigens useful in the diagnosis of leptospirosis by immunological methods and/or in the production of preventative and therapeutic vaccines.

### SUMMARY OF THE INVENTION

The inventive subject matter provides apparatus, systems and methods for the diagnosis, prevention, and treatment of leptospirosis. In one aspect, compositions comprising native and recombinant polypeptides of Leptospira interogans can be used for patient diagnosis. In another aspect, compositions comprising native and/or recombinant polypeptides of Leptospira interogans can be used as a preventative and/or therapeutic vaccine. Methods are also contemplated for enrichment analysis for selection of antigens, for identifying serodiagnostic or vaccine antigens and for producing diagnostic assays for antibodies to the proteins.

One embodiment of the inventive subject matter is an antigen composition that includes two or more antibody reactive antigens that are associated with a carrier. The antigens have known, quantified, and/or otherwise characterized reactivity (for example strength of interaction) with an antibody obtained from sera from a population exposed to or otherwise affected by leptospirosis. In some of such embodiments, the known antibody reactivity for such an antigen averages in the upper tertile of binding affinities of antibodies produced by a leptospirosis patient. Similarly, in other such embodiments of the inventive concept the average quantity of antibodies produced in a leptospirosis patient and directed to one or more such antigens is in the upper tertile. Similarly, in other such embodiments reactivities are characterized by an activity state of a leptospirosis infection. At least two of the antigens have a known association with a disease parameter, such as previous or current exposure to leptospirosis, an acute leptospirosis infection, a latent leptospirosis infection, a recurrent leptospirosis infection, a leptospirosis carrier state, and an at least partial immunity to infection with leptospirosis. Suitable antigens include LIC11352, LIC12544, LIC12631, LIC10464-s1, LIC11335, LIC20301, LIC10486, LIC10191, LIC11389, LIC11437, LIC20087, LIC10623, LIC10998, LIC10215, LIC11271, LIC10491-s1, LIC13050, LIC11210, LIC10524, LIC11456, LIC12476, LIC11570, LIC13244, LIC13238, LIC11885, LIC11008, LIC13242, LIC11336, LIC20250, LIC10525, LIC10464-s2.1, LIC20118, CopLigAU(unique):Repeats A7′-13, CopLigBU(unique):Repeats B7′-12, and CopLigB:Repeats 1-16, nt154-173 or fragments thereof. Such antigens or fragments thereof can be supplied as purified or at least partially purified proteins or peptides, for example having a purity of greater than 60%. At least two of such antigens of the compositions can be present in at least 40% of a population that is exposed to leptospirosis. In some embodiments of the inventive concept the carrier of the antigen composition is a pharmaceutical carrier, such as is used in the formulation of a vaccine (for example, a therapeutic vaccine). In an alternative embodiment, the carrier is an insoluble carrier upon which least two of the antigens are distinguishable from one another. Such insoluble carriers are useful in diagnostic assays, and include solid carriers with antigens disposed at separate and distinguishable locations (for example, in an array) and suspendable particles, where antigens are disposed upon different and distinguishable particle populations.

Another embodiment of the inventive subject matter is an antigen composition that is for use in diagnosing leptospirosis in a mammal, which includes two or more antibody reactive antigens that are associated with a carrier. The antigens have known, quantified, and/or otherwise characterized reactivity (for example strength of interaction) with an antibody obtained from sera from a population exposed to or otherwise affected by leptospirosis. In some of such embodiments, the known antibody reactivity for such an antigen averages in the upper tertile of binding affinities of antibodies produced by a leptospirosis patient. Similarly, in other such embodiments of the inventive concept the average quantity of antibodies produced in a leptospirosis patient and directed to one or more such antigens is in the upper tertile. Similarly, in other such embodiments reactivities are characterized by an activity state of a leptospirosis infection. At least two of the antigens have a known association with a disease parameter, such as previous or current exposure to leptospirosis, an acute leptospirosis infection, a latent leptospirosis infection, a recurrent leptospirosis infection, a leptospirosis carrier state, and an at least partial immunity to infection with leptospirosis. Suitable antigens include LIC11352, LIC12544, LIC12631, LIC10464-s1, LIC11335, LIC20301, LIC10486, LIC10191, LIC11389, LIC11437, LIC20087, LIC10623, LIC10998, LIC10215, LIC11271, LIC10491-s1, LIC13050, LIC11210, LIC10524, LIC11456, LIC12476, LIC11570, LIC13244, LIC13238, LIC11885, LIC11008, LIC13242, LIC11336, LIC20250, LIC10525, LIC10464-s2.1, LIC20118, CopLigAU(unique):Repeats A7′-13, CopLigBU(unique):Repeats B7′-12, and CopLigB:Repeats 1-16, nt154-173 or fragments thereof. Such antigens or fragments thereof can be supplied as purified or at least partially purified proteins or peptides, for example having a purity of greater than 60%. At least two of such antigens of the compositions can be present in at least 40% of a population that is exposed to leptospirosis. In some embodiments, the carrier is an insoluble carrier upon which least two of the antigens are distinguishable from one another. Such insoluble carriers are useful in diagnostic assays, and include solid carriers with antigens disposed at separate and distinguishable locations (for example, in an array) and suspendable particles, where antigens are disposed upon different and distinguishable particle populations. Alternatively, in some embodiments the carrier includes supendable particles that are distributed on a matrix (for example, a porous membrane or a fibrous sheet), where the matrix includes pores, interstitial spaces, or other openings that permit fluid to flow through the matrix.

Yet another embodiment of the inventive subject matter is an antigen composition for use as a leptospirosis vaccine in a mammal, where the antigen composition includes two or more antibody reactive antigens that are associated with a carrier. The antigens have known, quantified, and/or otherwise characterized reactivity (for example strength of interaction) with an antibody obtained from sera from a population exposed to or otherwise affected by leptospirosis. In some of such embodiments, the known antibody reactivity for such an antigen averages in the upper tertile of binding affinities of antibodies produced by a leptospirosis patient. Similarly, in other such embodiments of the inventive concept the average quantity of antibodies produced in a leptospirosis patient and directed to one or more such antigens is in the upper tertile. Similarly, in other such embodiments reactivities are characterized by an activity state of a leptospirosis infection. At least two of the antigens have a known association with a disease parameter, such as previous or current exposure to leptospirosis, an acute leptospirosis infection, a latent leptospirosis infection, a recurrent leptospirosis infection, a leptospirosis carrier state, and an at least partial immunity to infection with leptospirosis. Suitable antigens include LIC11352, LIC12544, LIC12631, LIC10464-s1, LIC11335, LIC20301, LIC10486, LIC10191, LIC11389, LIC11437, LIC20087, LIC10623, LIC10998, LIC10215, LIC11271, LIC10491-s1, LIC13050, LIC11210, LIC10524, LIC11456, LIC12476, LIC11570, LIC13244, LIC13238, LIC11885, LIC11008, LIC13242, LIC11336, LIC20250, LIC10525, LIC10464-s2.1, LIC20118, CopLigAU(unique):Repeats A7′-13, CopLigBU(unique):RepeatsB7′-12, and CopLigB:Repeats 1-16, nt154-173 or fragments thereof. Such antigens or fragments thereof can be supplied as purified or at least partially purified proteins or peptides, for example having a purity of greater than 60%. At least two of such antigens of the compositions can be present in at least 40% of a population that is exposed to leptospirosis. In some embodiments of the inventive concept the carrier of the antigen composition is a pharmaceutical carrier, such as is used in the formulation of a vaccine (for example, a therapeutic vaccine). Such a composition can additionally include an adjuvant.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 schematically depicts a method for generating a proteome array for identification of immunodominant Leptospira antigens.

FIG. 2 is a photomicrograph of a typical proteome microarray, interrogated using antibody to peptide markers present at all sites of the microarray.

FIG. 3 is a heatmap of observed signal intensities from a series of Leptospira proteome arrays interrogated with sera from healthy control individuals, individuals with acute leptospirosis, and convalescent phase leptospirosis, illustrating differential responses to a number of protein and peptide antigens.

FIG. 4A and FIG. 4B show signal intensity and BHp values for differential and for cross reactive antigens for sera from healthy individuals and acute phase leptospirosis patients (FIG. 4A) and for sera from healthy individuals and convalescent phase leptospirosis patients (FIG. 4B).

FIG. 5A and FIG. 5B shows results from interrogation of a series of Leptospira proteome arrays with sera from individuals in different control groups. FIG. 5A shows a heatmap of signal intensities from different individuals. FIG. 5B shows cumulative signal intensity for the different control groups as the number of antigens characterized increases.

FIG. 6A and FIG. 6B show ROC curves for various immunodominant antigens. FIG. 6A shows ROC curves for a number of individual antigens. FIG. 6B shows ROC curves for combinations of antigens.

FIG. 7 is a photograph of a series of immunoblots used to confirm Leptospira proteome array results.

### DETAILED DESCRIPTION

The inventive subject matter provides apparatus, systems and methods for the diagnosis, prevention, and treatment of leptospirosis. A microarray approach is utilized to display all or part of the proteome of Leptospira interogans, in order to permit interrogation of potential antigens of the organism with sera from infected populations in different phases of leptospirosis and with control sera from non-infected individuals. This permits simultaneous identification of specific antigens that invoke an immune response characteristic of a phase or stage of the disease and characterization of the degree of that response (for example, by comparing signal intensity to that of appropriate controls). Identification of characteristic antigens among populations that represent different stages of leptospirosis permits identification of protein antigens useful in diagnosis and staging of the disease. Characterization of the degree of immune response permits selection of antigens that invoke high affinity, high avidity, and/or highly expressed antibodies (i.e. immunodominant antigens) that are particularly useful in high sensitivity assays for leptospirosis and in vaccines for prevention or treatment of the disease. In one aspect, compositions comprising native and recombinant polypeptides of Leptospira interogans can be used for patient diagnosis. In another aspect, compositions comprising native and/or recombinant polypeptides of Leptospira interogans can be used as a preventative and/or therapeutic vaccine. Methods are also contemplated for enrichment analysis for selection of antigens, for identifying serodiagnostic or vaccine antigens and for producing diagnostic assays for antibodies to the proteins.

In other attempts to characterize human immune responses to leptospirosis, Inventors have found that the protein antigens studies are not randomly selected for recognition by the immune system. Characterizing the antibody response over thousands of potential antigens at the proteome scale allows molecular features related to antigenicity to be accurately classified. In addition, such array approaches permit rapid and convenient characterization of responses of different antibody classes and subclasses (ex: IgG, IgM, IgA, IgE, IgD) from the same set of characterized samples, and can be utilized to directly compare results from different leptospire species and serovars, and from different mammalian hosts. One should appreciate that the use of a proteome microarray advantageously permits identification of useful Leptospira interogans antigens that are not identified in prior art approaches that limit the range of potential antigens. Thus potentially useful antigens can be identified that are not identified, for example, by methods that focus on surface proteins. In addition, identification of immunodominant antigens permits utilization of Leptospira interogans antigens that do not merely invoke an antibody response, but rather invoke a strong and vigorous antibody response that is particularly useful in serological detection of leptospirosis and in vaccines that induce a protective or therapeutic immune responses in individuals exposed to Leptospira interogans.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The recitation of ranges of values herein is merely intended to serve as a shorthand method of referring individually to each separate value falling within the range. Unless otherwise indicated herein, each individual value with a range is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended.

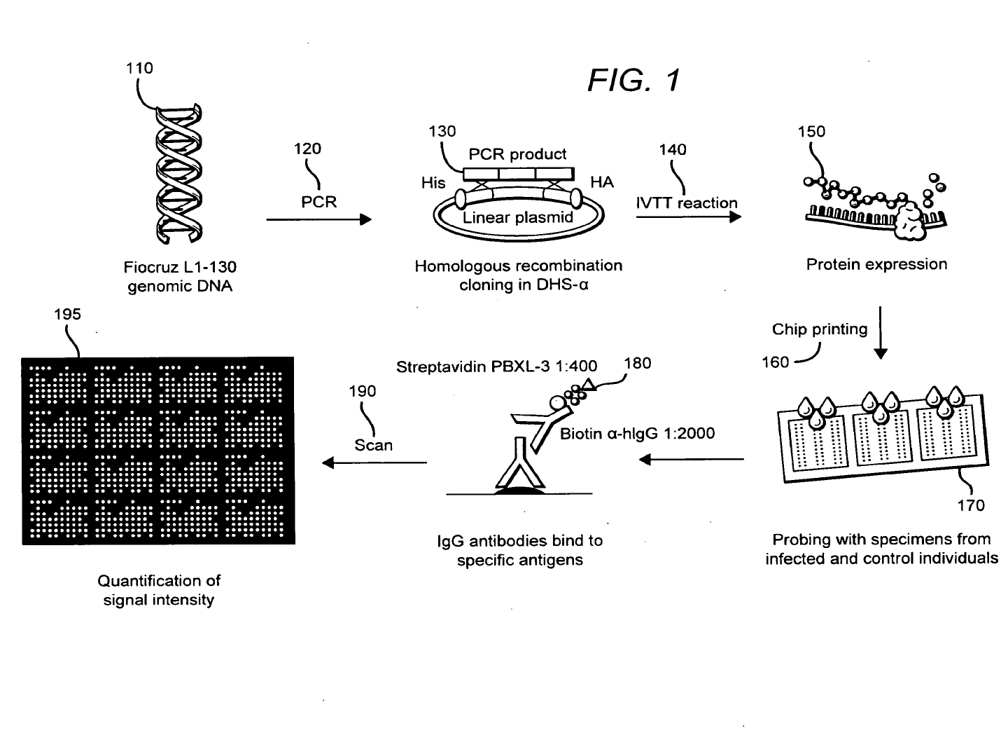

As noted above, in some embodiments of the inventive subject matter a proteome microarray can be used to identify immunodominant antigens associated with leptospirosis. Any suitable microarray format can be used, including planar microarrays, fluid or suspended microarrays, and microwell plates. A general approach to the use of a planar microarray is illustrated in FIG. 1. Details of an exemplary process are provided in the Examples below. As shown in FIG. 1, genomic DNA can be obtained from a Leptospira species 110 (in this instance, Leptospira interrogans serovar Copenhageni strain Fiocruz L1-130) and open reading frames (ORFs) amplified using PCR 120. In some embodiments, all ORFs are amplified, whereas in other embodiments PCR primers can be selected to amplify a subset of the available ORFs. For example, genomic data from sources such as the National Center for Biotechnology Information (NCBI) and John Craig Venter Institute (JCVI) can be used to identify ORFs that include proteins with potentially biological importance along with potentially antigenic features. PCR primers can be designed to include adapter sequences that permit insertion of PCR products into suitable cloning vectors 130, such as a linear plasmid. Such cloning vectors can include other useful sequences, such as sequences that code for peptide sequences that provide tags which permit subsequent isolation (for example, polyhistidine) and/or immunorecognition (for example, hemagglutinin). In some embodiments it can be necessary to split long ORFs into multiple segments, so that length of the ORF does not impede subsequent clonal expansion or expression. Following insertion into a suitable cloning vector, such vectors can be transferred to competent cells for expansion. Transformed cells can be identified by a suitable selection procedure (for example, antibiotic resistance), segregated, and the contents subjected to in vitro transcription 140 in order to produce proteins and/or peptides from each cloned ORF 150. Each of such individual proteins or peptides represents a potential antigen.

The potential antigens thus generated can then used to produce an array, for example through printing of individual in vitro transcription products at individual sites on a microarray chip 160. Such an array can also contain test sites that do not contain materials resulting from leptospiral ORFs, such as peptides associated with the vector (for example polyhistidine and hemagglutinin), control materials, or materials that are useful for the orientation and/or automated characterization of the array. It should be appreciated that an array can contain replicates of individual sites, which can be useful in permitting recovery from printing errors and in improving overall array performance. The array can then be probed or interrogated 170 with antibody-containing samples. Suitable samples include sera, plasma, or other fluids from infected individuals in different stages (i.e. acute, chronic, recovering, carrier) of leptospirosis and control samples from individuals from areas where leptospirosis is endemic or, alternatively, from where leptospirosis does not occur (i.e. naïve samples). Complex formation between antibodies from such samples and proteins or peptides within a test site can be visualized by interrogating the array with a secondary antibody binding agent 180 directed towards antibodies found in the samples (for example, an anti-IgG, anti-IgM, anti-IgA, anti-IgE, anti-IgD). Such secondary antibodies can carry a detectable “tag” which can be visualized directly (for example a fluorescent dye or protein), or indirectly (for example biotin or an enzyme). In embodiments utilizing secondary antibodies carrying tags that are visualized indirectly subsequent processing steps, for example incubation with labeled avidin/streptavidin or incubation with an enzyme substrate that generates a detectable product, can be carried out. It should be appreciated that the in vitro transcription process 140 can also generate non-relevant proteins and peptides that originate from the cells used to support the vector 130 in addition to proteins and peptides associated with the PCR amplified ORFs, and that it is possible that samples can contain antibodies directed to such non-relevant proteins and peptides. In such situations, samples can be treated with such non-relevant proteins (for example, cell lysates, products of in vitro translation processes that did not receive exogenous DNA, or products from in vitro translation processes following transformation with vectors that did not receive PCR products) prior to use in interrogating the array.

Following interrogation of the array with samples from appropriate individuals and visualization of antibody binding with a secondary antibody binding agent, results of the array study can be determined using an array scanner 190. The format and design of the array scanner is dependent upon the format and design of the array. For example, a fluid array of suspended and individually keyed particles can be scanned using a flow cytometer or similar device capable of identifying individual particles and measuring a signal (for example, fluorescence) from each that is characteristic of complex formation with antibody from a sample. Similarly, results from a study on a planar array can be scanned using a digital camera, microscope, optical scanner, or other image acquisition device and subsequent use of software that permits identification of individual test sites and measurement of a signal (for example, fluorescence, phosphorescence, luminescence. etc.) associated with complex formation with antibody from a sample. It should be appreciated that such planar arrays can be illuminated or excited via right angle, oblique angle, or epitaxial illumination during data acquisition, or, alternatively, can be generated on a substrate that acts as a one or two dimensional waveguide and can provide illumination or excitation via surface plasmon resonance. Alternatively, arrays produced using microwell plates can be characterized using a microwell plate reader. The signal representing complex formation between an antibody from a sample and a protein or peptide of the array can be quantified to provide a measure of the degree of antibody complex formation 195. For example, a small but statistically significant signal relative to that observed from a control sample or control site on an array can indicate that complex formation with an antigen of the array has occurred, but that the antibody affinity, avidity, or overall antibody response is relatively weak. Alternatively, a large signal relative to that observed from a control sample or site and/or to other identified antigens on the array can indicate that the antigen is an immunodominant antigen, i.e. an antigen that induces high affinity and/or high avidity antibodies, or that induces the formation of relatively high concentrations of antibodies. Alternatively, an antigen can be considered immunodominant if at least one of an average binding affinity, average binding avidity, and an average quantity of antibodies produced in a patient against the at least two antigens is in an upper tertile of binding affinity, binding avidity, and quantity of antibodies produced in a patient. Such immunodominant antigens are attractive targets for use in assays and in vaccines.

It should be appreciated that the basic method described in FIG. 1 has utility in identifying antigens or antigen panels that are useful both in the early diagnosis of leptospirosis (i.e. through utilizing antigens that provide a strong signal) and in the identification of specific stages or states of leptospirosis. For example, results of samples from acute cases of leptospirosis can be compared to results of samples from cases of recovering or convalescing patients in order to develop tests that aid the physician in diagnosing and treating this disease. Similarly, identifying immunodominant antigens characteristic of early stages and the late stages of leptospirosis can aid in the development of vaccines that are useful in the prevention and/or in the treatment of the disease by identifying antigenic materials that are likely to invoke a significant immune response. Such immunodominant antigens can be used individually or grouped as panels. Such panels can have utility in improving the sensitivity of an assay, in providing for accurate staging of the infection, and in providing protecting and/or therapeutic immunity. If the antigens in the combination were such that exclusive or non-exclusive licensure from only a single entity was required this would be attractive to diagnostic kit manufacturers who are discouraged from producing assays based on currently identified recombinant proteins because of the number of licenses that have to be obtained.

One contemplated embodiment of the inventive concept is an antigen composition can include a plurality of antibody reactive antigens associated with a carrier. At least two of the antigens can have (i) quantified and known relative antibody reactivities with respect to sera of a population affected by leptospirosis and (ii) a known association with a disease parameter (for example stage of the disease, duration of the disease, clinical outcome, and prior exposure). It is preferred that the plurality of antigens are selected from the group consisting of LIC11352, LIC12544, LIC12631, LIC10464-s1, LIC11573, LIC11335, LIC20301, LIC10486, LIC10191, LIC11389, LIC11437, LIC20087, LIC10623, LIC10998, LIC10215, LIC11271, LIC10491-s1, LIC13050, LIC11210, LIC10524, LIC11456, LIC12476, LIC11570, LIC13244, LIC13238, LIC11885, LIC11008, LIC13242, LIC11336, LIC20250, LIC10525, LIC10464-s2.1, LIC20118, LIC10973, LIC10464-s2, LIC10406, LIC12180, LIC11074, LIC10546, CopLigAU(unique):Repeats A7′-13, CopLigBU(unique):RepeatsB7′-12, and CopLigB:Repeats 1-16, nt154-1743, or fragments thereof.

It is contemplated that antigens of the inventive concept can have known reactivities, and that such known reactivities may be characterized by a variety of factors or parameters. It is, however, preferred that such known reactivities are characterized by strength of immunogenicity and/or time course of the Leptospiral infection. It is generally preferred that the parameter is activity state of the disease, a previous exposure to the pathogen, the duration of exposure to the pathogen, a chronic infection, past disease, active infection, inactive infection, at least partial immunity to infection with the pathogen, and/or outcome upon treatment. The disease parameter can be selected from the group consisting of a previous or current exposure to leptospirosis, acute, latent or recurrent infection, and at least partial immunity to infection with leptospirosis.

It is further contemplated that antigens of the inventive concept can have a characteristic distribution throughout a population. In contemplated embodiments, at least two of the antigens or antibodies to the at least two of the antigens can be present in at least 25%, 30%, 35%, 40%, 45%, 50%, 55%, 60%, 65%, 70%, 75%, 80%, 85%, 90%, or more than 90% of a population exposed to the at least two antigens. Optionally, at least one of an average binding affinity and an average quantity of antibodies produced in a patient against the at least two antigens is in an upper tertile of binding affinity and quantity of antibodies produced in a patient.

In some embodiments of the inventive concept, antigens identified using the described methods (or fragments thereof) and/or antibodies directed to them can be utilized in diagnosis of leptospirosis in a mammal and/or in a diagnostic device or system suitable for diagnosis of leptospirosis in a mammal. In a similar embodiment, antigens identified using the described methods (or fragments thereof) and/or antibodies directed to them can be utilized in a method for diagnosing leptospirosis in a mammal. Such antigens can be used individually. In a preferred embodiment 2, 3, 4, 5, 6, 7, 8, 9, 10, 11, 12, 13, 14, 15, 16, 17, 18, 19, 20, or more than 20 of such antigens or antibodies directed to such antigens can be used as a panel for testing purposes. Such protein or peptide antigens can be recombinant and can be at least partially purified. The purity of protein or peptide antigens used can be 10%, 20%, 30%, 40%, 50%, 60%, 70%, 80%, 90%, 95%, 98%, 99%, or greater than 99% on a w/w basis. A diagnostic device or method can be directed towards identification of leptospirosis, staging of leptospirosis, or both. The diagnostic device or method can use any suitable immunoassay format, including agglutination, nephelometry, radioimmunoassay, enzyme immunoassay, fluorescence anisotropy, and immunofluorescence. Direct, indirect, or “sandwich” assay methods or formats can be used. Suitable diagnostic devices and methods include flow assays, microwell plate assays, and lateral flow assays. In such assays one or more components are bound or coupled to a carrier such as a solid or insoluble phase (e.g. a plastic surface, a glass surface, a paper or fibrous surface, or the surface of a particle). In assays utilizing multiple antigens, such antigens or antibodies to such antigens can be distributed on the test surface or surfaces so as to be distinguishable from one another. For example, individual antigens or antibodies can be fixed to different wells of a microwell plate or to distinguishable and/or discrete sites on a planar test surface. Alternatively, individual antigens or antibodies to individual antigens can be coupled to different populations of particles that are distinguishable by color, size, and/or fluorescence emission. In still other embodiments of the inventive concept, the diagnostic device is a flow or strip test, in which the flow of a fluid through a support that includes passages, openings, or channels for flow (for example a porous membrane or a fibrous sheet) moves test components past reactive sites. In such an embodiment identification of individual antigens or antibodies to individual antigens can be ascertained through the appearance of an indicator (for example, a colored band or stripe) at a characteristic position on the support.

In another embodiment, a method of predicting a likelihood of a patient having leptospirosis can include determining autoantibody reactivity against one or more antigens, or their variants, in a sera sample obtained from a patient. A likelihood of the patient having leptospirosis can then be predicted from reference samples derived from sera of patients diagnosed as having the disease, such that increased or decreased autoantibody reactivity against selected antigens can be positively correlated with increased likelihood of the disease in the patient.

Another embodiment of the inventive concept is a vaccine preparation that includes immunogenic amounts one or more antigens (or fragments thereof) identified using sera from individuals with leptospirosis, in combination with a carrier (such as a physiologically acceptable vehicle). In a similar embodiment, one or more antigens (or fragments thereof) of the inventive concept can be used in a method for treating and/or preventing leptospirosis through use in a vaccine. Such a vaccine can be prophylactic and/or therapeutic. In a preferred embodiment a vaccine preparation includes 2, 3, 4, 5, 6, 7, 8, 9, 10, 11, 12, 13, 14, 15, 16, 17, 18, 19, 20, or more than 20 Leptospira antigens, at least some of which are immunodominant antigens. Such protein or peptide antigens can be recombinant and can be at least partially purified. The purity of the protein or peptide antigens used can be 40%, 50%, 60%, 70%, 80%, 90%, 95%, 98%, 99%, or greater than 99% on a w/w basis. Such vaccines can also include effective amounts of immunological adjuvants, known to enhance an immune response. To immunize a subject, the immunogenic protein(s) or peptide(s) can be administered parenterally, usually by intramuscular or subcutaneous injection. Other modes of administration, however, are also acceptable. For example, the vaccine may be administered orally, or via a mucosal route, such as a nasal, gastrointestinal or genital site. Suitable vaccine formulations contain an effective amount of the active ingredient in a vehicle. The effective amount in a prophylactic vaccine is an amount sufficient to prevent, ameliorate, reduce the incidence of Leptospira infection in the target mammal. The effective amount in a therapeutic vaccine is an amount sufficient to reduce bacterial load, reduce symptoms, and or shorten the course of a Leptospira infection in the target mammal. The effective amount is readily determined by one skilled in the art. The active ingredient may typically range from about 1% to about 95% (w/w) of the composition, or even higher or lower if appropriate. The quantity to be administered depends upon factors such as the age, weight, and physical condition of the vaccinated subject. A vaccine of the inventive subject can be administered in a single dose or in multiple doses, as required for desired effectiveness.

Protein or peptide antigens identified as described for use in a vaccine can be isolated, lyophilized, and stabilized prior to vaccine preparation. The vaccine may then be adjusted to an appropriate concentration, optionally combined with a suitable vaccine adjuvant, and packaged for use. Typical adjuvants include surfactants (e.g. hexadecylamine, octadecylamine, lysolecithin, dimethyldioctadecylammonium bromide, N,N-dioctadecyl-N′-N-bis(2-hydroxyethyl-propane di-amine), methoxyhexadecyl-glycerol, and pluronic polyols), polyanions (e.g., pyran, dextran sulfate, poly IC, polyacrylic acid, carbopol), peptides (e.g., muramyl dipeptide, MPL, aimethylglycine), tuftsin, oil emulsions, alum, and mixtures thereof. Such immunogenic product may be encapsulated into liposomes for use in a vaccine formulation, or may be conjugated to proteins such as keyhole limpet hemocyanin (KLH) or human serum albumin (HSA) or other polymers.

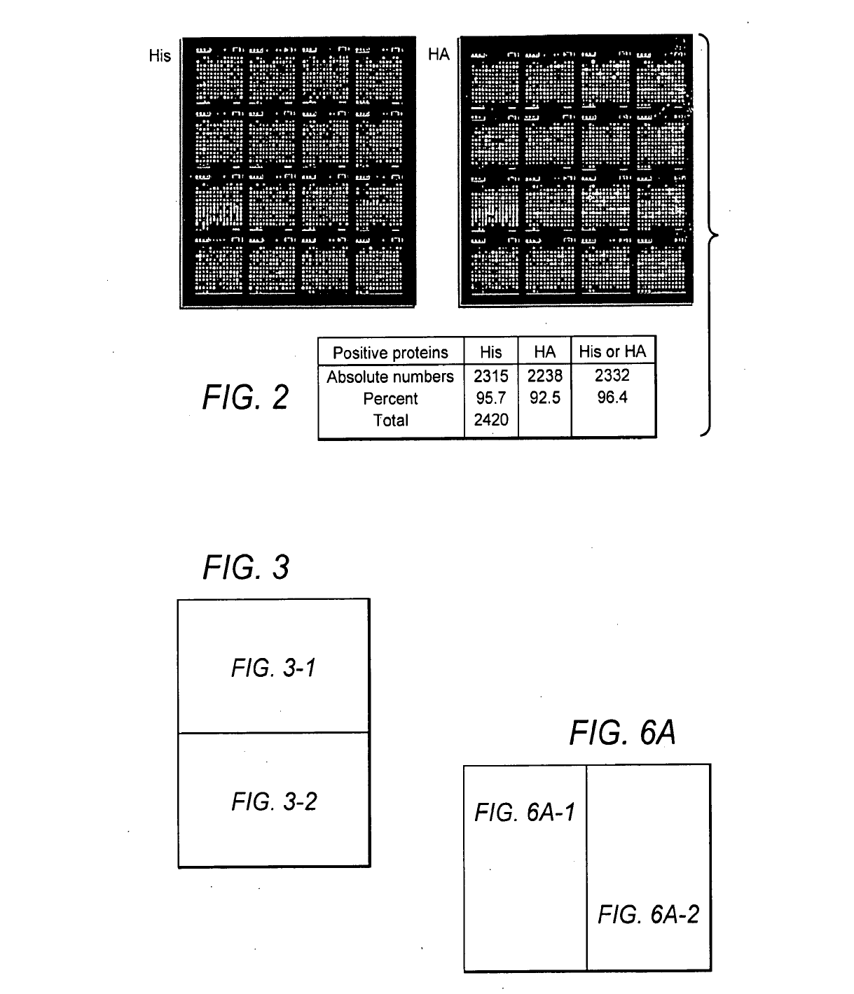

In one aspect of the inventive subject, the genomic approaches described were applied to construct point-of-care tests to diagnose leptospirosis. Accordingly, a protein microarray chip was developed comprising 61% of the Leptospira interrogans serovar Copenhageni strain Fiocruz L1-130 genome. Chip fabrication involved a 3 step process: (1) PCR amplification of each selected ORF, (2) in vivo recombination cloning and (3) in vitro transcription-translation reaction followed by the microarray chip printing. Proteins were expressed with both polyhistidine (His) and hemagglutinin (HA) tags, which permitted antibodies directed to these tags to be used to assess microarray chip quality. As shown in FIG. 2, 96% of the printed test sites were positive for either tag.

Results from such microarrays were evaluated using a signal intensity of greater than 2.5 standard deviations above the average intensities of test sites generated from products of control (i.e. performed without DNA) reactions as a cutoff for significant antibody complex formation. Sera of laboratory confirmed cases for leptospirosis in Salvador/Brazil as well as different sets of negative controls were probed for complex formation with IgG. A set of 23 differentially reactive (p<10-4) immunodominant antigens was identified among convalescent samples (n=80) when compared to control, of which 16 were also differentially reactive (p<0.02) among acute samples (n=50). For 8 of these 16 and for 3 of the remaining 7 antigens in this set, an increasing reactivity (p<0.04) was detected from acute to convalescent phase, suggesting that these antigens might be related to protective immunity. It is notable that some of these proteins, such as LipL32 and the unique domains of the Leptospira immunoglobulin-like proteins LigA and LigB, had been previously described as sero-reactive antigens, providing validation for this approach. Leptospira antigens identified by such an embodiment are listed in Table 1.

TABLE 1

| | Acute group | | | Convalescent group | | |
|---|---|---|---|---|---|---|
| | Average | | | Average | | |
| | signal | BHp- | Reactivity | signal | BHp- | Reactivity |
| Antigen ID | intensity | value | classification | intensity | value | classification |
| LIC11352 | 25150.3 | 8.54E−11 | DR | 42950.1 | <1E−14 | DR |
| LigA Repeats 7-13 | 13002.0 | 8.54E−11 | DR | 27995.7 | <1E−14 | DR |
| LigB Repeats 7-12 | 9824.4 | 1.80E−06 | DR | 22508.6 | <1E−14 | DR |
| LigA/B Repeats 1-6 | 8351.1 | <1E−14 | DR | 12572.6 | <1E−14 | DR |
| LIC20042 | 7718.2 | 7.07E−03 | DR | 3991.3 | 8.68E−01 | CR |
| LIC10191 | 7585.8 | 2.07E−03 | DR | 6210.0 | 5.52E−02 | CR |
| LIC11335 | 7497.6 | 1.40E−04 | DR | 7122.7 | 1.14E−05 | DR |
| LIC10215 | 6427.4 | 1.42E−11 | DR | 3804.5 | 1.79E−13 | DR |
| LIC11389 | 6149.7 | 2.49E−04 | DR | 5692.5 | 2.73E−06 | DR |
| LIC11955 | 5858.7 | 2.75E−03 | DR | — | — | — |
| LIC11573 | 5838.1 | 1.73E−05 | DR | 9287.4 | <1E−14 | DR |
| LIC11222 | 5694.5 | 4.01E−04 | DR | — | — | — |
| LIC10486 | 5687.5 | 6.57E−03 | DR | 6896.8 | 5.04E−09 | DR |
| LIC11271 | 4300.9 | 9.08E−04 | DR | 4919.1 | 3.83E−14 | DR |
| LIC12180 | 3982.6 | 5.85E−03 | DR | — | — | — |
| LIC11456 | 3191.7 | 2.69E−05 | DR | — | — | — |
| LIC10483 | 8018.0 | 2.31E−01 | CR | 10707.6 | 6.23E−04 | DR |
| LIC11437 | 5182.2 | 6.36E−02 | CR | 5434.7 | 3.77E−05 | DR |
| LIC12544 | 10205.5 | 1.03E−01 | CR | 16651.7 | <1E−14 | DR |
| LIC10502-s4 | 11161.0 | 1.78E−01 | CR | 6979.2 | 7.57E−01 | CR |
| LIC10902 | 10708.7 | 8.46E−01 | CR | 12698.5 | 3.69E−01 | CR |
| LIC11019 | 8317.5 | 6.56E−01 | CR | 6862.7 | 5.45E−01 | CR |
| LIC11052 | 4182.3 | 1.23E−01 | CR | 3964.1 | 7.84E−01 | CR |
| LIC11834 | 8927.6 | 8.95E−01 | CR | 6081.0 | 9.61E−01 | CR |
| LIC13050 | 4265.3 | 3.45E−01 | CR | 3597.8 | 1.52E−01 | CR |
| LIC13084 | 9030.7 | 1.44E−01 | CR | 8420.3 | 9.77E−01 | CR |
| LIC20218 | 3666.6 | 8.94E−01 | CR | 3516.4 | 9.59E−01 | CR |
| LIC10090 | 3764.8 | 8.11E−01 | CR | — | — | — |
| LIC11089 | 4943.0 | 2.19E−01 | CR | — | — | — |
| LIC11210 | 3532.1 | 1.31E−01 | CR | — | — | — |
| LIC11486 | 3799.1 | 9.71E−01 | CR | — | — | — |
| LIC12270 | 4089.8 | 4.73E−01 | CR | — | — | — |
| LIC12895 | 4117.8 | 2.99E−01 | CR | — | — | — |
| LIC13392 | 3339.9 | 4.84E−01 | CR | — | — | — |
| LIC20112 | 3032.1 | 9.30E−01 | CR | — | — | — |
| LIC20301 | — | — | — | 6546.6 | 8.83E−12 | DR |
| LIC20087 | — | — | — | 4660.7 | <1E−14 | DR |
| LIC10623 | — | — | — | 3571.0 | 2.19E−02 | DR |
| LIC11570 | — | — | — | 3233.2 | 2.21E−14 | DR |
| LIC10524 | — | — | — | 3054.3 | 1.40E−08 | DR |
| LIC10053 | — | — | — | 3081.8 | 8.80E−01 | CR |
| LIC11122 | — | — | — | 3301.9 | 7.87E−01 | CR |

DR=differentially reactive; CR=cross-reactive when compared to healthy individuals from high endemic area group. Blanks correspond to antigens that were either differentially or cross-reactive for one group but the average signal intensity was below the cut-off for the other group.
   Products associated with individual antigens are shown in Table 2.

TABLE 2

| Antigen ID | Product |
|---|---|
| LIC11352 | LipL32 - lipoprotein 32 |
| LigA Repeats 7-13 | LigA - immunoglobulin-like protein A |
| LigB Repeats 7-12 | LigB - immunoglobulin-like protein B |
| LigA/B Repeats 1-6 | LigA/B - immunoglobulin-like protein A/B |
| LIC20042 | BatC |
| LIC10191 | Loa22 - peptidoglican associated protein |
| LIC11335 | GroEL - chaperonin |
| LIC10215 | hypothetical protein LIC10215 |
| LIC11389 | FlbB - flagelar protein B |
| LIC11955 | hypothetical protein LIC11955 |
| LIC11573 | GspG - general secretory pathway protein G |
| LIC11222 | hypothetical protein LIC11222 |
| LIC10486 | hypothetical protein LIC10486 |
| LIC11271 | hypothetical protein LIC11271 |
| LIC12180 | methyltransferase |
| LIC11456 | LipL31 - lipoprotein 31 |
| LIC10483 | hypothetical protein LIC10483 |
| LIC11437 | adenylate/guanylate cyclase |
| LIC12544 | DNA binding protein |
| LIC10502-s4 | cytoplasmic membrane protein |
| LIC10902 | hypothetical protein LIC10902 |
| LIC11019 | putative lipoprotein |
| LIC11052 | hypothetical protein LIC11052 |
| LIC11834 | putative lipoprotein |
| LIC13050 | hypothetical protein LIC13050 |
| LIC13084 | hypothetical protein LIC13084 |
| LIC20218 | hypothetical protein LIC20218 |
| LIC10090 | hypothetical protein LIC10090 |
| LIC11089 | hypothetical protein LIC11089 |
| LIC11210 | hypothetical protein LIC11210 |
| LIC11486 | hypothetical protein LIC11486 |
| LIC12270 | hypothetical protein LIC12270 |
| LIC12895 | putative lipoprotein |
| LIC13392 | polysaccharide deacetylase |
| LIC20112 | periplasmic protein, LipL45 homologue |
| LIC20301 | hypothetical protein LIC20301 |
| LIC20087 | outer membrane protein |
| LIC10623 | flagellar motor protein MotB |
| LIC11570 | GspD - general secretory pathway protein D |
| LIC10524 | DnaK - molecular chaperone |
| LIC10053 | hypothetical protein LIC10053 |
| LIC11122 | putative lipoprotein |

Utilizing the proteome array approach described herein and interrogating the proteome arrays with samples from natural human infections with Leptospira interrogans, the following antigens (listed in Table 3) have also been shown to be immunodominant antigens.

TABLE 3

| Locus Tag | Description | Symbol |
|---|---|---|
| CopLigAU(unique):RepeatsA7′-13 | | |
| CopLigBU(unique):RepeatsB7′-12 | | |
| LIC12631 | hemolysin | |
| LIC10464-s1 | | |
| CopLigB:Repeats1-16, nt154-1743 | | |
| LIC10998 | hypothetical protein LIC10998 | |
| LIC12476 | dihydrolipoamide succinyltransferase | sucB |
| LIC13244 | isocitrate dehydrogenase | icdA |
| LIC13238 | hypothetical protein LIC13238 | |
| LIC11885 | putative lipoprotein | |
| LIC11008 | hypothetical protein LIC11008 | |
| LIC13242 | response regulator | |
| LIC11336 | co-chaperonin GroES | groES |
| LIC20250 | peptidoglycan-associated cytoplasmic | |
| | membrane protein | |
| LIC10525 | heat shock protein GrpE | grpE |
| LIC10464-s2.1 | | |
| LIC20118 | hypothetical protein LIC20118 | — |
| LIC10973 | outer membrane protein | ompL1 |
| LIC10464-s2 | | |
| LIC10406 | hypothetical protein LIC10406 | |
| LIC11074 | hypothetical protein LIC11074 | — |
| LIC10546 | lipase | |

It should be appreciated that the proteome approach described herein identified a number of heretofore unknown proteins as immunodominant antigens associated with vigorous immune responses to leptospirosis. Surprisingly, a number of Leptospira proteins were identified that are not expressed on the surface of organism and would not be considered as potential antigenic sites using conventional approaches.

As noted above, one embodiment of the inventive concept is a composition having partially purified and/or recombinant proteins of leptospirosis disease Leptospira interrogans species proteins for use in methods such as diagnostic tests, in a variety of different formats, for antibodies to several antigens in which the antigens are alone or in combination with one or more other recombinant proteins. Such a diagnostic assay can be for antibodies to Leptospira interrogans or, alternatively, another leptospirosis disease Leptospira species. While such a composition, device, or method can be used for laboratory support of the diagnosis of leptospirosis, it can also be useful for staging the infection and/or for assessing the outcome of antibiotic therapy. As shown in Table 4, single antigens identified using the proteome microarray can, for example, be used to differentiate between acute-phase and convalescent-phase patients with leptospirosis. While results with single antigens are shown, it is contemplated that results with 2, 3, 4, 5, 6, 7, 8, 9, 10, 11, 12, 13, 14, 15, 16, 17, 18, 19, 20, or more than 20 Leptospira antigens can provide more accurate and/or sensitive results.

TABLE 4

| | Acute | | | | Convalescent | | | |
|---|---|---|---|---|---|---|---|---|
| Antigen | Se | Spe | AUC | BHp-value | Se | Spe | AUC | BHp-value |
| LigA7-13 | 88.0% | 87.5% | 0.894 | <1E−14 | 94.0% | 98.8% | 0.965 | <1E−14 |
| LigB7-12 | 86.0% | 83.8% | 0.857 | 8.54E−11 | 96.0% | 96.3% | 0.968 | <1E−14 |
| LIC11352 | 88.0% | 73.8% | 0.841 | 8.54E−11 | 92.0% | 97.5% | 0.986 | <1E−14 |
| LIC10215 | 92.0% | 67.5% | 0.865 | 1.42E−11 | 86.0% | 83.8% | 0.879 | 1.79E−13 |
| LIC11573 | 80.0% | 67.5% | 0.775 | 1.73E−05 | 88.0% | 88.8% | 0.926 | <1E−14 |
| LIC11456* | 82.0% | 68.8% | 0.763 | 2.69E−05 | 80.0% | 78.8% | 0.851 | 1.57E−09 |
| LigA/B | 74.0% | 72.5% | 0.785 | 1.80E−06 | 86.0% | 96.3% | 0.956 | <1E−14 |
| LIC11335 | 92.0% | 53.8% | 0.724 | 1.40E−04 | 90.0% | 62.5% | 0.749 | 1.14E−05 |
| LIC11222* | 94.0% | 50.0% | 0.711 | 4.01E−04 | 94.0% | 30.0% | 0.568 | 4.33E−01 |
| LIC11389 | 60.0% | 77.5% | 0.737 | 2.49E−04 | 66.0% | 77.5% | 0.770 | 2.73E−06 |
| LIC11955* | 90.0% | 45.0% | 0.687 | 2.75E−03 | 90.0% | 20.0% | 0.477 | 9.67E−01 |
| LIC11271 | 88.0% | 46.3% | 0.713 | 9.08E−04 | 88.0% | 78.8% | 0.877 | 3.83E−14 |
| LIC10486 | 90.0% | 42.5% | 0.677 | 6.57E−03 | 90.0% | 67.5% | 0.831 | 5.04E−09 |
| LIC12180* | 86.0% | 45.0% | 0.663 | 5.85E−03 | 88.0% | 27.5% | 0.534 | 5.36E−01 |
| LIC10191* | 36.0% | 93.8% | 0.678 | 2.07E−03 | 90.0% | 43.8% | 0.655 | 5.52E−02 |
| LIC20042* | 82.0% | 45.0% | 0.672 | 7.07E−03 | 22.0% | 91.3% | 0.525 | 8.68E−01 |
| LIC20087* | 76.0% | 60.0% | 0.699 | 3.32E−03 | 96.0% | 86.3% | 0.948 | <1E−14 |
| LIC12544* | 88.0% | 41.3% | 0.615 | 1.03E−01 | 88.0% | 91.3% | 0.917 | <1E−14 |
| LIC11570* | 86.0% | 56.3% | 0.750 | 3.15E−04 | 96.0% | 78.8% | 0.892 | 2.21E−14 |
| LIC20301* | 86.0% | 51.3% | 0.698 | 2.77E−03 | 92.0% | 75.0% | 0.861 | 8.83E−12 |
| LIC10524* | 78.0% | 76.3% | 0.781 | 1.59E−05 | 78.0% | 81.3% | 0.846 | 1.40E−08 |
| LIC11437* | 82.0% | 45.0% | 0.633 | 6.36E−02 | 74.0% | 72.5% | 0.767 | 3.77E−05 |
| LIC10483* | 68.0% | 50.0% | 0.575 | 2.31E−01 | 68.0% | 67.5% | 0.715 | 6.23E−04 |
| LIC10623* | 96.0% | 11.3% | 0.456 | 6.05E−01 | 86.0% | 43.8% | 0.670 | 2.19E−02 |

Se = Sensitivity; Spe = Specificity; AUC = Area under the curve.
*Antigens in italic/bold were considered either not seroreactive (average signal intensity below the cut-off, italic) or cross-reactive (BHp < 0.05, bold) for that group but were among the differentially reactive set for the other group.

Contemplated methods also include enrichment analysis for selecting antigens. Using the systems and methods described herein, it is contemplated that over 90% of serodiagnostic antigens from ⅓ of the Leptospira interrogans genome can be identified.

### EXAMPLES

### Methods

Human Study Protocols:

Protocols were approved by the institutional review board committees of Yale University and Oswaldo Cruz Foundation. Samples were obtained from infected patients and healthy individuals living in a community with high endemic transmission of leptospirosis and participants provided written informed consent. Blood donors from the city of Salvador were anonymous. Sera from U.S. healthy individuals were obtained from anonymous volunteers at the General Clinical Research Center at the University of California, Irvine. After collection, a code number was designated to each patient so that all samples were anonymized prior to use.

Human Samples:

Evaluations were performed with a collection of 114 control human serum samples and 160 laboratory-confirmed sera of leptospirosis cases. Control samples were (i) 29 sera from healthy volunteers from California/US, where endemic transmission of leptospirosis does not exist; (ii) 35 sera from blood donors from Salvador/Brazil, city with endemic transmission of leptospirosis and (ii) 50 sera from healthy subjects who were enrolled in a cohort study in a high risk urban slum community in the same city. Cases were identified during active hospital-based surveillance in the same state of the slum community, including patients from the city of Salvador and from the country side, from April 1996 to August 2010. During this period, 1529 MAT-confirmed cases of severe leptospirosis were identified, of which we selected 80 acute- and 80 convalescent-phase sera to conduct this study. Serum samples were randomly selected and therefore acute and convalescent samples are not necessarily paired. Acute-phase samples were collected upon patient admittance at the hospital and convalescent-phase samples were collected from recovering patients at least 14 days after hospital admittance and that may or may not have received standard antibiotic therapy. Laboratory confirmation was defined according to the criteria for seroconversion, a four-fold rise in titer or a single titer of 1:800 in the MAT.

Microarray Target Selection:

Selection of the open reading frames (ORFs) that would compose the array was performed considering the Leptospira interrogans serovar Copenhageni strain Fiocruz L1-130 genome annotations available at National Center for Biotechnology Information (NCBI) and at John Craig Venter Institute (JCVI) databases. The criteria used included proteins with potentially biological importance along with potentially antigenic features.

PCR Amplification and High Throughput Recombination Cloning:

Selected open reading frames (ORFs) were amplified by PCR and cloned into pXI vectors using a high-throughput PCR recombination cloning method. Briefly, ORFs were amplified using 5 ng of L. interrogans serovar Copenhageni strain Fiocruz L1-130 with Accuprime Taq DNA Polimerase System (Invitrogen, Grand Island, N.Y., USA) according to the manufacturer's protocol. Cycling conditions were as follows: 94° C. for 2 minutes, 31 cycles of 94° C. for 90 seconds, 55° C. for 15 seconds, 50° C. for 15 seconds, 68° C. for 2 minutes, and a final extension at 68° C. for 10 minutes. Primers contained a 20 bp ORF-specific sequence and a unique 20 bp adapter sequence, which becomes incorporated into the 59 and 39 termini flanking the amplified gene and is homologous to the cloning sites of the linearized pXI vector (ACGACAAGCATATGCTCGAG and TCCGGAACATCGTATGGGTA, respectively). Genes larger than 3 kb were cloned as smaller segments, maintaining an overlap of at least 150 nucleotides between the sequences. Such segmented ORFs were named with the gene ID followed by the letter “s” and the number of the segment, e.g. LIC10502-s4. The ligA and ligB genes (LIC10465 and LIC10464, respectively) were fragmented with respect to the repeated Big domains present in the protein's structures (LigB Repeats 7-12, LigA Repeats 7-13 and LigA/B Repeats 1-6) [20], which have been described as potential diagnostic markers and/or vaccine candidates. Up to three additional rounds of amplification were attempted for failures, which were generally recovered by adjusting the PCR conditions. All PCR reactions were confirmed for correct insert size by gel electrophoresis prior to cloning.

The pXI plasmid encodes an N-terminal 6×His-tag and a C-terminal hemagglutinin (HA) tag. The plasmid was linearized by digestion with BamH1 and amplified by PCR to generate the acceptor vector. A reaction containing 40 ng of linearized pXI vector, 1 μL of ORF PCR reaction and 10 μL of super-competent Escherichia coli DH5-a cells (McLab) was incubated on ice for 30 minutes, heat-shocked at 42° C. for 1 minute and chilled on ice for 1 min. One hundred and eighty μL of S.O.C medium were added and cells were cultured for 1 hour at 37° C. The entire reaction mixture was added to 1.1 mL of LB supplemented with kanamycin (50 μg/mL) and incubated overnight at 37° C. with vigorous aeration. Plasmids were extracted with QIAprep 96 Turbo Kit (Qiagen, Valencia, Calif. USA) without colony selection and analyzed by gel electrophoresis to confirm insert size. Up to 2 additional rounds of cloning were performed to increase efficiency and were resumed by doubling the PCR volume for transformation. All plasmids carrying inserts of less than 500 bp and some randomly selected ones were confirmed for insert presence by PCR using the insert specific primers. After probing the microarrays with the serum samples, the seroreactive antigens were identified and the corresponding plasmids were sequenced. The insert was confirmed in all cases.

Microarray Fabrication and Interrogation:

For array fabrication, purified minipreparations of DNA were used for expression in an E. coli based in vitro transcription/translation (IVTT) reaction system (RTS Kit, Roche Applied Science, Indianapolis, Ind., USA) according to the manufacturer's instructions. Ten μL reactions were performed in 384-well plates and incubated for 16 hours at 26° C. under 300 rpm shaking. Control reactions were performed in the absence of DNA (“NoDNA” controls) to assess the background given by the IVTT reaction itself. Protease inhibitor mixture (Complete, Roche Applied Science, Indianapolis, Ind., USA) and Tween-20 to a final concentration of 0.5% v/v were added to the reactions, which were then mixed and centrifuged to pellet any precipitates and remove bubbles prior to printing. The crude supernatants were immediately printed onto nitrocellulose coated glass FAST slides (Whatman, Piscataway, N.J., USA) using an Omni Grid 100 microarray printer (Genomic Solutions, Cambridgeshire, UK). In addition, arrays were printed with multiple negative control reactions, positive control spots of an IgG mixture containing mouse, rat and human IgGs (Jackson ImmunoResearch, West Grove, Pa., USA) and purified Epstein-Barr Virus Nuclear Antigen 1 (EBNA1) protein, which is recognized by the majority of humans (allowing it to serve as a marker for serum quality).

Protein expression was verified by probing the array with monoclonal anti-polyhistidine tag (Sigma Aldrich, St. Louis, Mo., USA) and anti-hemaglutinin tag (Roche Applied Science, Indianapolis, Ind., USA) against the respective tags. Arrays were initially blocked for 30 minutes with Protein Array Blocking Buffer (Whatman, Piscataway, N.J., USA) and probed overnight with anti-tag antibodies diluted 1:400 in Blocking Buffer. Arrays were then incubated for one hour in biotinylated secondary antibodies (Jackson ImmunoResearch, West Grove, Pa., USA) diluted 1:1000 in Blocking Buffer followed by a one hour incubation with streptavidin-conjugated SureLight P3 (Columbia Biosciences, Frederick, Md., USA). After each incubation, slides were washed 3 times with Tris buffered saline containing Tween-20 at 0.05% v/v (TTBS). Additional washes with Tris buffered saline (TBS) and distilled water were performed and the slides were air-dried by brief centrifugation before scanning. Slides were scanned in a Perkin Elmer ScanArray confocal laser (Perkin Elmer, Waltham, Mass., USA) and intensities were quantified using QuantArray software (Packard Biochip Technologies, Billerica, Mass., USA).

For interrogation with human serum, samples were diluted 1:100 in Protein Array Blocking Buffer containing 10 mg/mL E. coli lysate (McLab, San Francisco, Calif., USA) at a final concentration of 10% v/v and incubated for 30 minutes at room temperature under constant mixing to remove background reactivity to E. coli proteins in the IVTT reactions. E. coli protein-antibody complexes were removed from the sample dilution mix via centrifugation prior to addition to the microarray. Arrays were blocked for 30 minutes with Protein Array Blocking Buffer and then incubated with diluted samples overnight at 4° C. with gentle rocking. Biotinylated anti-human immunoglobulin G (Fc-c fragment specific, Jackson ImmunoResearch, West Grove, Pa., USA) was diluted 1:2000 in Blocking Buffer and incubated with the arrays for one hour incubation at ambient temperature. Slides were washed 3 times with TTBS after each incubation and bound antibodies were detected by our incubation with streptavidin-conjugated SureLight P3 for one hour, as described above. Following incubation with SureLight P3, microarray results were quantified by were scanning for fluorescence intensity.

Microarray Data Analysis:

Spot intensities were quantified using QuantArray software (Packard Biochip Technologies, Billerica, Mass., USA). Raw data were obtained as the mean pixel signal intensity for each spot and all intensities were automatically corrected for spot specific background. For each array, the average of control IVTT reactions (i.e. the No DNA controls) was subtracted from spot signal intensities in order to minimize background reactivity. Proteins were considered to be expressed when signal intensity for either of the tags was greater than the No DNA control reactions mean plus 2.5 standard deviations. The same cut-off was applied to identify reactive proteins interrogated using the collected serum samples. Data analysis was performed using publically available R statistical software (obtained at http://www.r-project.org). To stabilize the variance, VSN normalization was applied to the raw data and groups were compared by a Bayes regularized t test. The Benjamini and Hochberg (BH) method was used to control the false discovery rate so that a p-value smaller than 0.05 was considered significant and the corresponding protein was considered differentially reactive. For histograms, BH corrected p-values smaller than 1 E-14 were assigned as 1 E-16. Multiplex classifiers were generated using linear and nonlinear Support Vector Machines (SVMs) using the “e1071” R statistical software. Plots of receiver operating characteristic (ROC) curves were made with the “ROCR” R statistical software. Sensitivity and specificity were determined from the resulting ROC curves. Clinical characteristics of the leptospirosis patients whose acute and/or convalescent serum samples were selected for this study were described using frequencies and medians with interquartile (IQR) ranges. The Chi square test or the Mann-Whitney/Wilcoxon test was used to compare clinical presentations of acute-phase leptospirosis patients with convalescent-phase patients. An association between patients' clinical characteristics and the intensity of acute sera signal against the three antigens that presented the best performance in the protein microarray were evaluated by the Kruskal-Wallis test.

Immunostrip Blotting:

Selected clones, corresponding to differentially reactive antigens for either acute or convalescent sample groups, were submitted to a five-hour IVTT reaction (RTS Kit, Roche Applied Science, Indianapolis, Ind., USA) according to the manufacturer's instructions. Protease inhibitor mixture (Complete, Roche Applied Science, Indianapolis, Ind., USA), Tween-20 and methanol were added to final concentrations of 0.5% and 10% v/v respectively. The reactions were mixed and centrifuged to remove bubbles. Unpurified supernatants were printed onto Hi-Flow Plus HF240 membrane (Millipore, Billerica, Mass., USA) using a BioJet dispenser (BioDot, Irvine, Calif., USA) at 1 μL/cm and cut into 3 mm membrane strips. Individual membrane strips were then blocked in TTBS 5% non-fat milk for 30 min. Sera samples were diluted 1:250 in TTBS 5% nonfat milk containing E. coli lysate at a final concentration of 20% v/v and incubated for 30 minutes at room temperature under agitation. Blocked strips were then incubated with diluted sera during 1 hour and washed 6 times with TTBS. Alkaline phosphatase-conjugated anti-human IgG (Jackson ImmunoResearch, West Grove, Pa., USA) was diluted 1:5000 in TTBS 5% nonfat milk and applied to each strip for 1 hour at room temperature under agitation. After washing 6 times with TTBS, 3 additional washes with TBS were performed and reactive bands were visualized by incubation with 1-step Nitro-Blue Tetrazolium Chloride/5-Bromo-4-Chloro-39-Indolyphosphate p-Toluidine Salt (NBT/BCIP) developing buffer (Thermo Fisher Scientific, Waltham, Mass., USA) for 2 minutes at room temperature. The enzymatic reaction was halted by exposing the membrane strips to running tap water and the membrane strips were air-dried before scanning at 2,400 dpi using a commercial desktop scanner (Hewlett-Packard, Palo Alto, Calif., USA). Images were converted to gray scale and band intensities were quantified using the ImageJ software package (available at http://rsbweb.nih.gov/ij/).

Exemplary Results

Antigen Selection:

The criteria used to select proteins for inclusion in the array provided 2,241 ORFs, which corresponded to 61% of the Leptospira interrogans proteome. In total, the array contained 2,361 antigens, including full length proteins and protein segments. Protein expression was evaluated by probing the array with anti-His and anti-HA, and over 97% of protein spots were confirmed positive for either His or HA tags.

Human IgG Antibody Profiles:

Sera used in this study were classified into 5 groups, summarized in Table 1 and described in the methods section. The majority of sera (88%) were obtained from male subjects and the median age was 34 (IQR: 24-45) years old. Median duration of symptoms before hospitalization was 6 (IQR: 5-8) days. Jaundice and acute respiratory distress syndrome occurred in 87% and 13% of the patients, respectively. Renal impairment was frequent (median creatinine: 4.0 [IQR: 2.0-6.4] mg/dL) and 30% of the patients received peritoneal or hemodialysis. Intensive care was provided for 20% of the patients and 3% died.

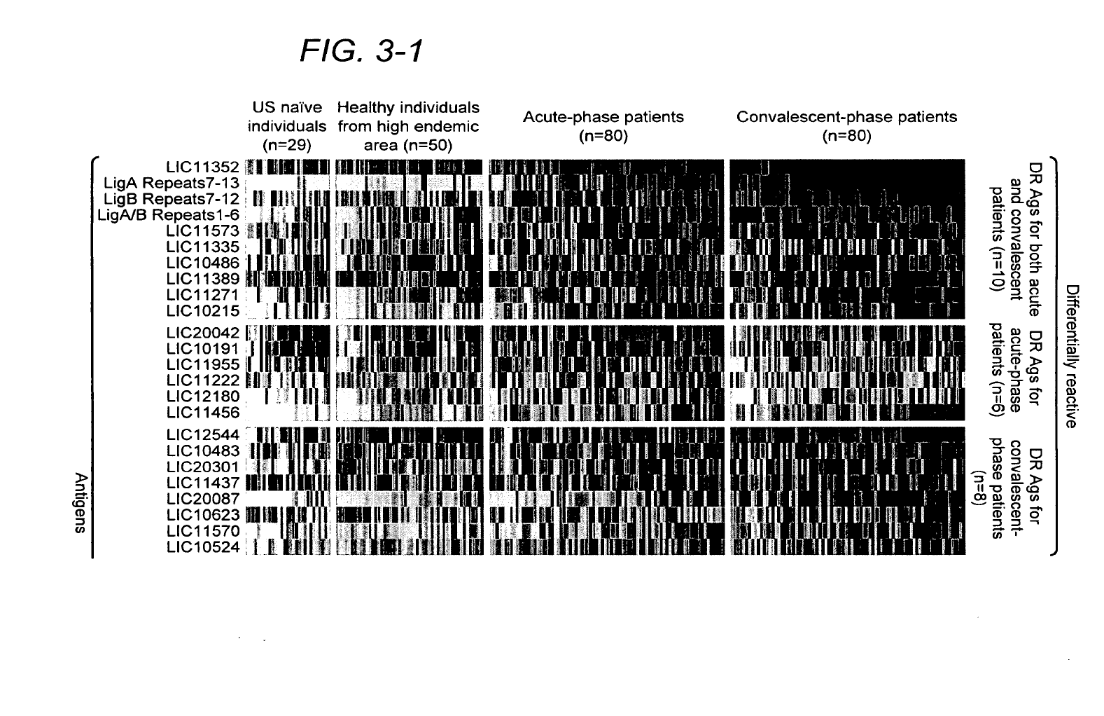

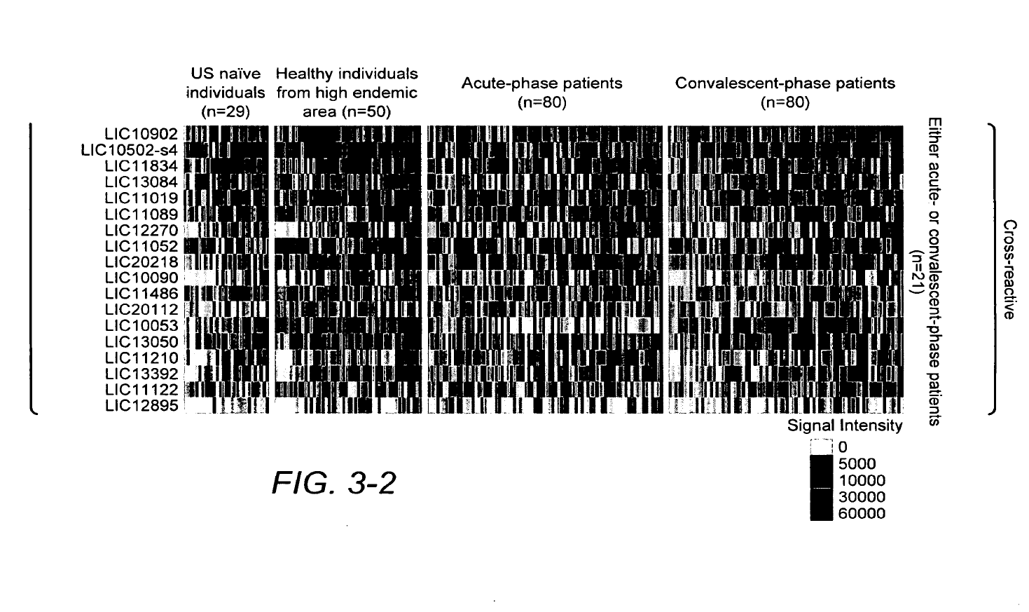

A heatmap summarizing array results is shown in FIG. 3, and gives an overview of the reactivity of the 42 reactive antigens for each of 239 individual samples. Individual specimens are in columns and grouped by healthy controls from USA, healthy controls from the high endemic area group, acute-phase patients and convalescent phase patients. The antigens, in rows, are organized according to those that are significantly more reactive in the cases than in the healthy controls. These antigens are termed ‘differentially reactive’ (DR) and are divided in 3 sections: antigens identified as differentially reactive for both acute- and convalescent-phase patients, antigens identified as differentially reactive only for acute patients and differentially reactive antigens only for convalescent patients. A second set of antigens were identified as equally as reactive in healthy controls and the cases, and termed ‘cross-reactive’ (CR). The background reactivity seen from the cross-reactive antigens was similar between all three groups.

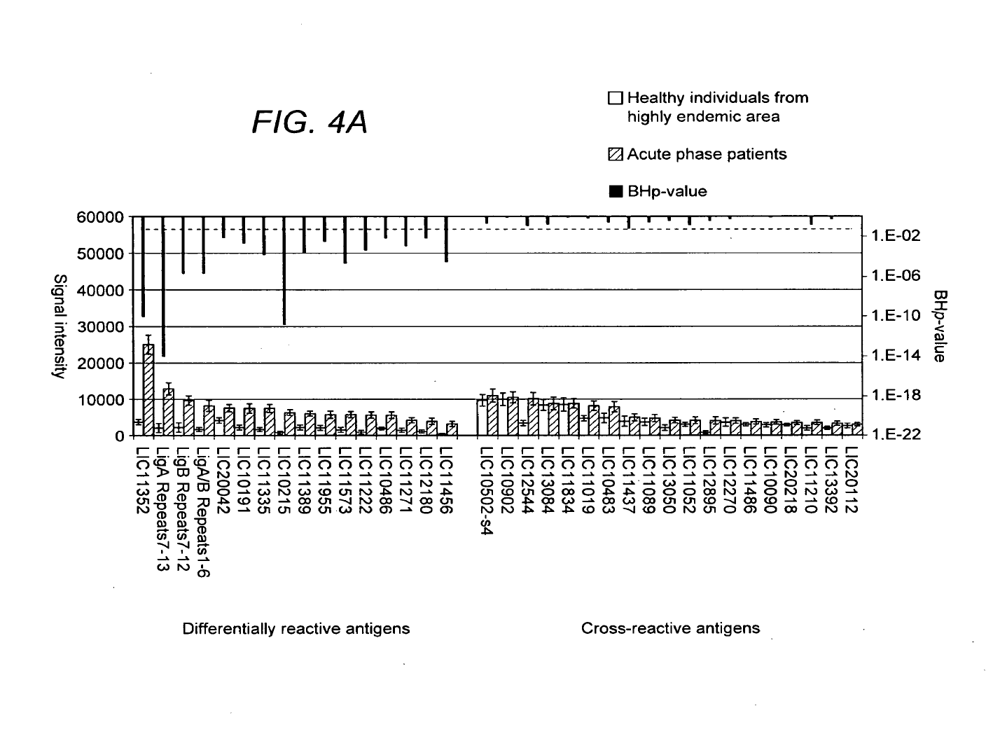

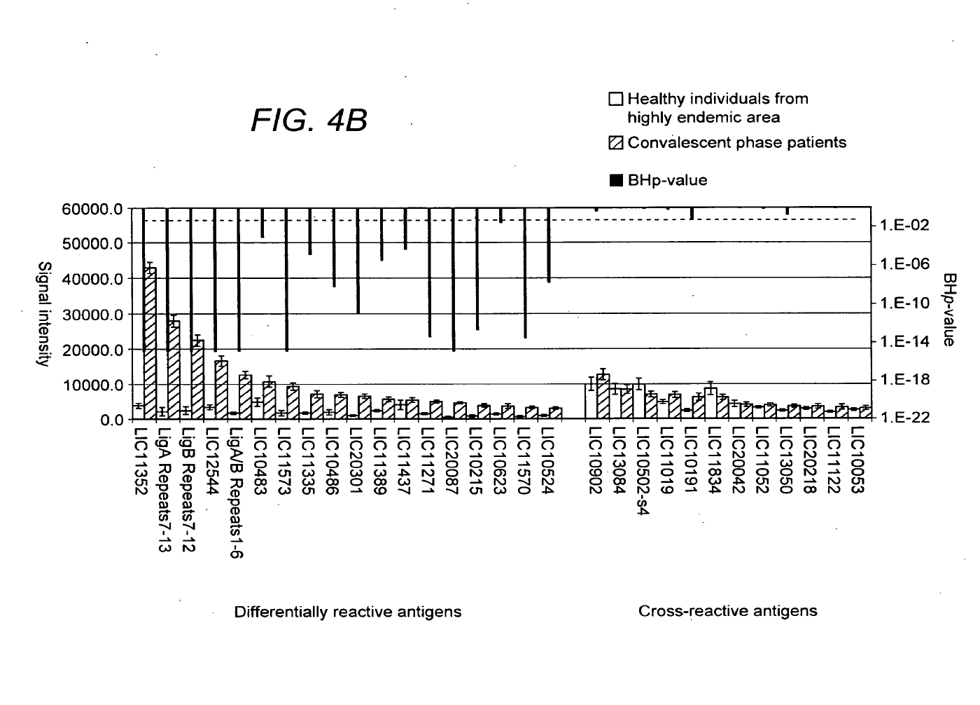

Towards the end of identifying antigens that can discriminate between positive and negative leptospirosis cases, analysis can be based on comparing acute and convalescent-phase patients to healthy individuals from an area with high endemic transmission. Results of such a comparison can be seen in FIG. 4A and FIG. 4B. Since healthy individuals living such an area can show some background reactivity to leptospiral lipopolysaccharides and proteins, identification of antigens with sero-reactivity among leptospirosis patients but not among those healthy individuals can be useful in distinguishing a current leptospirosis case. All the high endemic controls used in this sample set were MAT-negative for leptospirosis. In order to avoid bias in the analysis, the IgG reactivity detected on the microarray from samples obtained from 10 MAT-positive and 10 MAT-negative healthy individuals living in the collection area were compared. The overall reactivity observed for both groups was low and majority of the reactive antigens detected for infected patients (as described below) were not reactive for either MAT-positive or MAT-negative healthy individuals. Therefore MAT-negative high endemic controls were used for analysis.

This sample set identified 30 antigens, approximately 1.3% of all of the antigens printed on the array, as reactive antigens. Of these, 18 bound significantly more IgG antibody from the convalescent samples compared to control individuals from the high endemic area group. In acute-phase samples the IgG antibody response identified 35 seroreactive antigens or 1.5% of the array, of which 16 discriminated between acute and negative cases. LipL32, LigA Repeats 7-13 and LigB Repeats 7-12 antigens were the three most reactive targets on average for both convalescent- and acute phase groups. Ten differentially reactive antigens were identified as overlapping (i.e. in common) between the acute and convalescent groups.

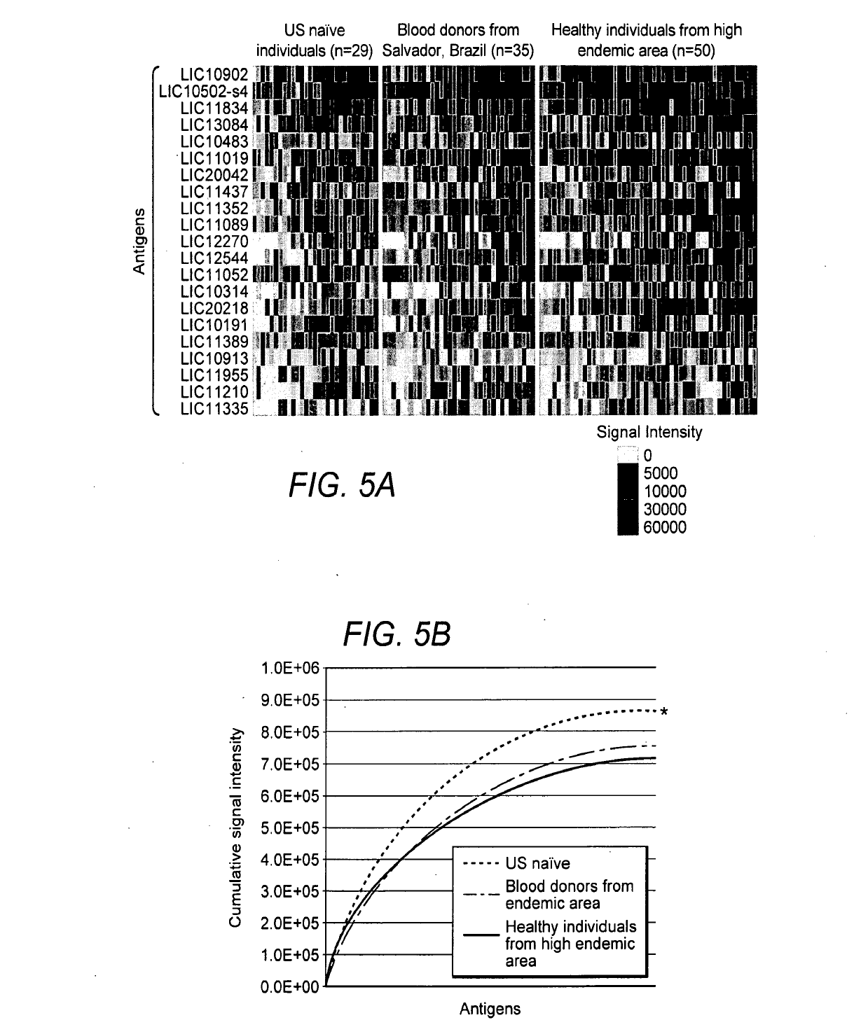

In order to characterize background reactivity among healthy individuals living in an area with endemic transmission of leptospirosis, cumulative antigen reactivity for control groups from the USA (where leptospirosis is not endemic), Brazilian blood donors, and healthy individuals from the high endemic area were compared. A heatmap of typical results is shown in FIG. 5A, which displays the reactivity of all antigens with an average signal intensity above the cut-off for any of the control groups. Higher overall reactivity was observed in the high endemic area group compared to USA controls and Brazilian blood donors. Analysis of the cumulative signal intensity against all antigens on the array (FIG. 5B) shows USA healthy subjects that are naïve to leptospirosis exposure showed the lowest total reactivity, followed by blood donors from Salvador and then healthy individuals from high endemic area. Blood donors living in endemic area had slightly higher reactivity than USA naïve subjects, but the difference observed with the exemplary sample set was not statistically significant. However, the total background reactivity in healthy individuals residing in the area with high endemic transmission was significantly greater (p<0.05) than either the blood donors from Brazil or the USA controls.

Average signal intensity for all the reactive antigens for each patient was also compared to the patient's MAT titer. MAT results are primarily dependent upon agglutinating antibodies that bind to leptospiral LPS, and do not differentiate between IgM and IgG immunoglobulin subtypes. All acute and convalescent samples were laboratory confirmed for infection by MAT, a 3-fold increase was observed in the median titer for convalescent samples compared samples from the acute group (i.e., from 800 to 3,200). a general increase in antigen signal intensities for the convalescent group compared to acute group was observed (see Figure XXX1 and XXX2), it was not possible to draw a correlation between these two approaches using this sample set. While not wishing to be bound by theory, Inventors feels that this may indicate that MAT antigen and protein antigens identify different antibody pools in these patient populations.

ROC Analysis of Serodiagnostic Antigens:

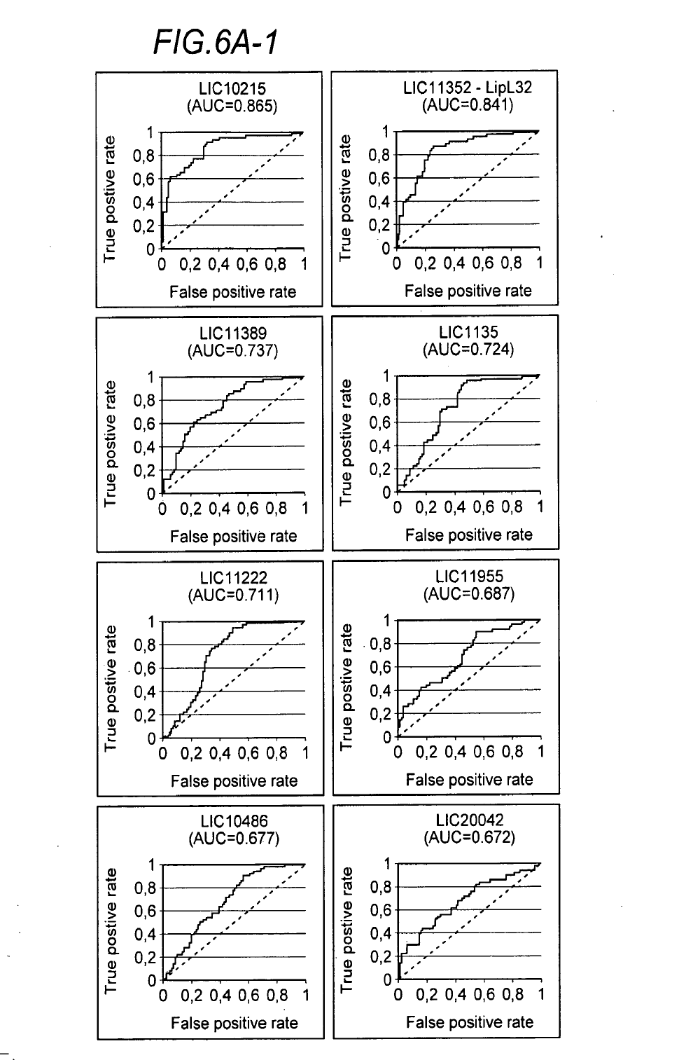

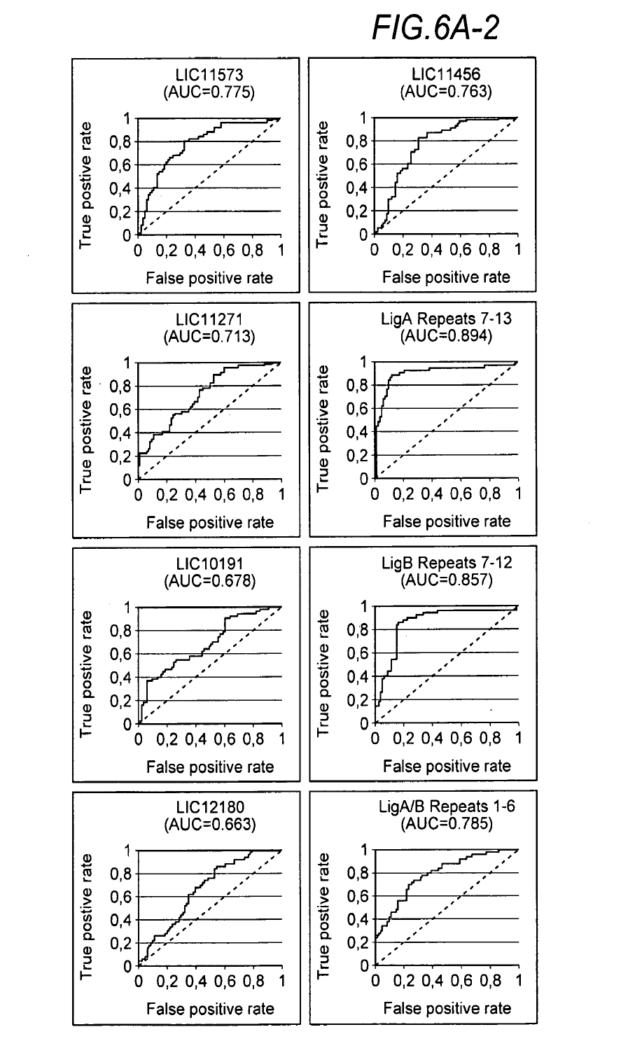

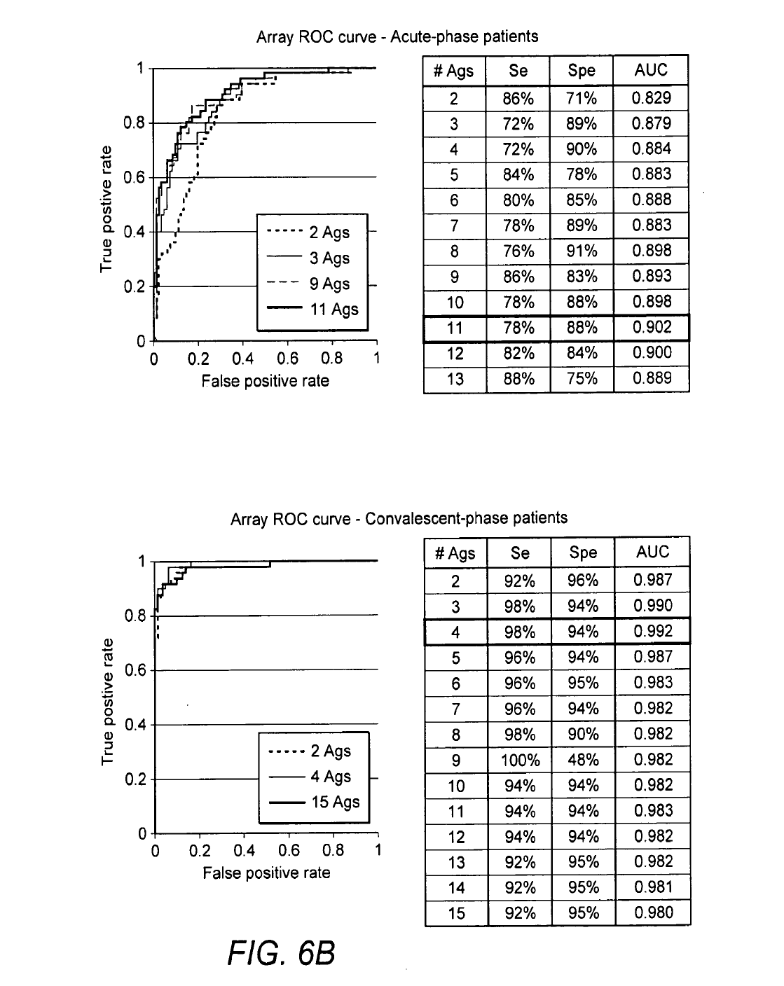

To determine the accuracy of the differentially reactive antigens in distinguishing a leptospirosis case, individual antigen ROC curves were generated and the AUC for each antigen was determined. Acute and convalescent-phase samples were analyzed separately against the high endemic area control group and sensitivity and specificity were calculated for both groups using the SVM computational approach. Antigens were then ranked by decreasing AUC and multiple antigens ROC curves generated. Exemplary single antigen ROCs for acute-phase group are shown in FIG. 6A; similar calculations were performed for samples from the convalescent-phase group. For both groups, the false positive rate was calculated using data from a high endemic area healthy control group.

For acute-phase patients, the non-identical domains of the Lig proteins (LigA Repeats 7-13 and LigB Repeats 7-12) provided best sensitivity and specificity (AUC=0.894-0.857), followed by LipL32 (LIC11352, AUC=0.841, Table 2). As disease progresses to convalescence, the accuracy of these antigens increases so that LipL32 achieves best performance (AUC=0.986) followed by LigA Repeats 7-13 (AUC=0.965) and LigB Repeats 7-12 (AUC=0.968). None of the three antigens with improved accuracy (i.e. LigA Repeats 7-13, LigB Repeats 7-12 and LipL32) were found to have high signal intensities when tested with acute serum sample (as associated patient's clinical characteristics). A heat shock protein of the GroEL family (LIC11335) was also identified as seroreactive, with high sensitivity for both acute- and convalescent-phase patients (90.0% and 92.0%, respectively) but low specificity (53.8% and 62.5%). DnaK (LIC10524), another heat shock protein, showed seroreactivity for the convalescent group, although we could not detect significant levels of IgG against this antigen in the acute group. The virulence-associated protein Loa22 (LIC10191) showed very low sensitivity for acute-phase patients (36.0%) and was considered not seroreactive for the convalescent group. Similarly, the IgG response against LipL31 (LIC11456) was detected only among acute patients, with a diagnostic accuracy of 82% sensitivity and 68.8% specificity.

Surprisingly, several novel antigens, for which no seroreactivity had been previously described, were identified by the Inventors. The hypothetical protein LIC10215 provided 92.0% and 86.0% sensitivity and 67.5% and 83.8% specificity for distinguishing healthy from either acute- or convalescent-phase patients, respectively. LIC10215 was found to be highly useful for distinguishing an acute case from a healthy individual after the domains of the Lig proteins and LipL32. Regarding the convalescent group, LIC20087, antigen annotated as outer membrane protein, provided the best accuracy after the domains of the Lig proteins and LipL32, with 96.0% sensitivity and 86.3% specificity.

The novel arrayed antigen approach utilized herein also permitted the unanticipated finding that the combination of 11 differentially reactive antigens allowed for excellent sensitivity and specificity for detection of acute leptospirosis cases (78.0% and 87.5%, respectively), whereas a combination of 4 antigens provided best accuracy (98.0% sensitivity and 94.0% specificity) for convalescent cases (see FIG. 6B).

Array Validation by Immunoblotting:

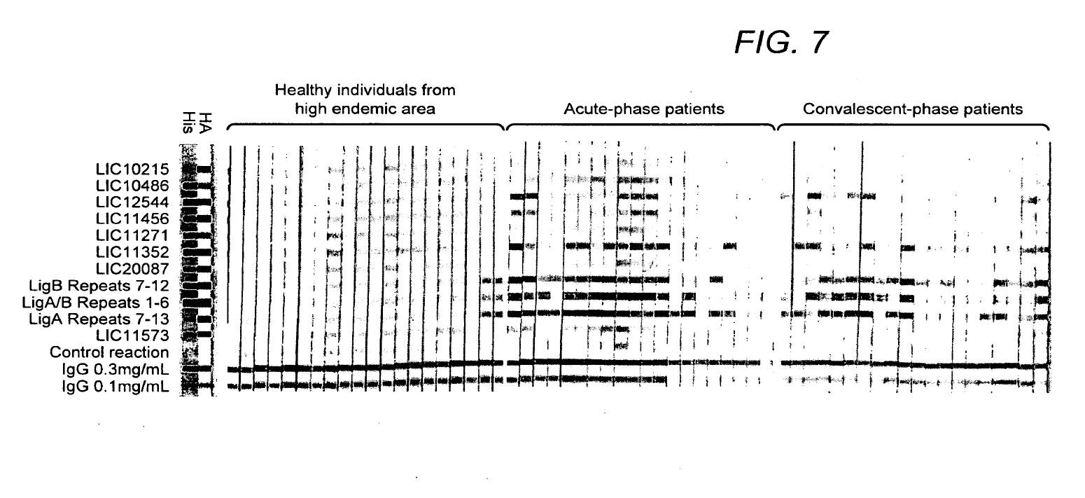

Eleven differentially reactive antigens, corresponding to the most significant antigens for either acute- or convalescent-phase groups were printed onto a nitrocellulose membrane and cut into 3 mm strips (immunostrips) which were interrogated with sera from 20 healthy control from highly endemic area, 20 acute leptospirois, and 20 convalescent leptospirosis individuals that were randomly selected. Healthy individuals showed lower reactivity against these antigens whereas leptospirosis patients reacted strongly against most of the antigens (FIG. 7). Antigen intensities were quantified and groups were compared using Bayes regularized t-test adapted from Cyber-T. For both acute- and convalescent-phase groups, the domains of the Lig proteins provided the best single antigen discrimination, followed by LipL32. Antigens LIC10215, LIC10486, LIC11271, LIC20087 and LIC11573 showed no sero-reactivity on immunostrips. Inventors hypothesize that the lower reactivity observed for these protein antigens on immunostrips may be due to technical differences between the immunostrip and array platforms.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1. An antigen composition comprising:
a plurality of antibody reactive antigens associated with or formulated with a carrier,
wherein at least two of the antibody reactive antigens have quantified and known relative antibody reactivities with respect to sera of a population affected by leptospirosis,
and wherein the at least two of the antibody reactive antigens have a known association with a disease parameter;
wherein the plurality of antibody reactive antigens are selected from the group consisting of:
LIC1 1352, LIC12544, LIC12631, LIC10464-s1, LIC1 1335, LIC20301, LIC10486, LIC10191, LIC1 1389, LIC1 1437, LIC20087, LIC10623, LIC10998, LIC10215, LIC1 1271, LIC10491-s1, LIC13050, LIC1 1210, LIC10524, LIC1 1456, LIC12476, LIC1 1570, LIC13244, LIC13238, LIC1 1885, LIC1 1008, LIC13242, LIC1 1336, LIC20250, LIC10525, LIC10464-s2.1, LIC20118, Cop LigAU (unique):Repeats A7′-13, CopLigBU (unique):Repeats B7′-12, CopLigB:Repeats 1-16, nt154-173 and antibody reactive fragments thereof.

2. The antigen composition of claim 1, wherein:
(a) the known relative antibody reactivities are characterized by strength of interaction with an antibody;
(b) the known relative antibody reactivities are characterized by an activity state of leptospirosis,
(c) the known relative antibody reactivities are characterized by (a) and (b),
(d) the disease parameter is selected from the groups consisting of: a previous or current exposure to leptospirosis, an acute leptospirosis infection, a latent leptospirosis infection, a recurrent leptospirosis infection, a leptospirosis carrier state, and an at least partial immunity to infection with leptospirosis;
(e) the at least two of the plurality of antibody reactive antigens are present in at least 40% of a population exposed to leptospirosis;
(f) the known relative antibody reactivity comprises an average antibody binding affinity in the upper tertile of binding affinities of antibodies produced in a leptospirosis patient; or
(g) the antigen composition of (f), wherein an average quantity of antibodies produced in a leptospirosis patient and directed against the at least two antigens is in an upper tertile of the antibodies produced in the leptospirosis patient.

3-7. (canceled)

8. The antigen composition of claim 1, wherein:
(a) the carrier is a pharmaceutically acceptable carrier;
(b) the antigen composition is formulated as a vaccine;
(c) the antigen composition of (b), wherein the vaccine is a therapeutic vaccine;
(d) the antigen composition comprises at least four antibody reactive antigens;
(e) the carrier is an insoluble carrier;
(f) at least two of the plurality of antigens are distinguishable when disposed upon the carrier;
(g) the carrier is a solid carrier and a first antigen of the at least two of the plurality of antibody reactive antigens is disposed upon a first location of the solid carrier, and a second antibody reactive antigen of the at least two of the plurality of antibody reactive antigens is disposed upon a second location of the solid carrier, and the first location is distinguishable from the second location;
(h) the carrier comprises suspendable particles, and a first antibody reactive antigen of the at least two of the plurality of antigens is disposed upon a first suspendable particle, a second antibody reactive antigen of the at least two of the plurality of antibody reactive antigens is disposed upon a second suspendable particle, and the first suspendable particle is distinguishable from the second suspendable particle;
(i) at least one of the antibody reactive antigens, or antibody reactive fragments thereof, are at least partially purified;
(j) at least one of the antibody reactive antigens or antibody reactive fragments thereof are recombinant; or
(k) at least one of the antibody reactive antigens or antibody reactive fragments thereof are present in a purity of greater than about 60%.

9-16. (canceled)

17. An antigen composition for use in diagnosing leptospirosis in a mammal, comprising:
a plurality of antibody reactive antigens associated with a carrier,
wherein at least two of the antibody reactive antigens have quantified and known relative antibody reactivities with respect to sera of a population affected by leptospirosis, and wherein the at least two of the antibody reactive antigens have a known association with a disease parameter;
wherein the plurality of antigens are selected from the group consisting of: LIC1 1352, LIC12544, LIC12631, LIC10464-s1, LIC1 1335, LIC20301, LIC10486, LIC10191, LIC1 1389, LIC1 1437, LIC20087, LIC10623, LIC10998, LIC10215, LIC1 1271, LIC10491-s1, LIC13050, LIC1 1210, LIC10524, LIC1 1456, LIC12476, LIC1 1570, LIC13244, LIC13238, LIC1 1885, LIC1 1008, LIC13242, LIC1 1336, LIC20250, LIC10525, LIC10464-s2.1, LIC20118, Cop LigAU (unique):Repeats A7′-13, CopLigBU(unique):Repeats B7′-12, CopLigB:Repeats 1-16, nt154-173 and antibody reactive fragments thereof.

18. The antigen composition of claim 17, wherein:
(a) the known reactivities are characterized by strength of interaction with an antibody;
(b) the known reactivities are characterized by an activity state of leptospirosis;
(c) the disease parameter is selected from the groups consisting of a previous or current exposure to leptospirosis, an acute leptospirosis infection, a latent leptospirosis infection, a recurrent leptospirosis infection, a leptospirosis carrier state, and an at least partial immunity to infection with leptospirosis;
(d) the at least two of the antibody reactive antigens are present in at least 40% of a population exposed to leptospirosis;
(e) the known relative antibody reactivity comprises an average antibody binding affinity in the upper tertile of binding affinities of antibodies produced in a leptospirosis patient;
(f) an average quantity of antibodies produced in a leptospirosis patient and directed against the at least two antigen is in an upper tertile of the antibodies produced in the leptospirosis patient;
(g) the carrier is an insoluble carrier, and wherein at least two of the plurality of antibody reactive antigens are distinguishable when disposed upon the carrier;
(h) the carrier is a solid carrier, and a first antibody reactive antigen of the at least two of the plurality of antigens is disposed upon a first location of the solid carrier, and a second antibody reactive antigen of the at least two of the plurality of antibody reactive antigens is disposed upon a second location of the solid carrier, and wherein the first location is distinguishable from the second location;
(i) the carrier comprises suspendable particles and a first antibody reactive antigen of the at least two of the plurality of antigens is disposed upon a first suspendable particle, and a second antibody reactive antigen of the at least two of the plurality of antibody reactive antigens is disposed upon a second suspendable particle, and wherein the first suspendable particle is distinguishable from the second suspendable particle;
(j) the carrier comprises suspendable particles disposed upon a matrix, and wherein the matrix comprises a plurality of openings that permit fluid flow through the matrix; or
(k) at least one of the antibody reactive antigens or antibody reactive fragments thereof are at least partially purified and/or are recombinant; or
(l) at least one of the antigens or fragments thereof are present in a purity of greater than 60%.

19-30. (canceled)

31. An antigen composition for use as a leptospirosis vaccine in a mammal, comprising:
a plurality of antibody reactive antigens associated with a carrier,
wherein at least two of the antibody reactive antigens have quantified and known relative antibody reactivities with respect to sera of a population affected by leptospirosis,
and wherein the at least two of the antibody reactive antigens have a known association with a disease parameter;
wherein the plurality of antibody reactive antigens are selected from the group consisting of LIC1 1352, LIC12544, LIC12631, LIC10464-s1, LIC1 1335, LIC20301, LIC10486, LIC10191, LIC1 1389, LIC1 1437, LIC20087, LIC10623, LIC10998, LIC10215, LIC1 1271, LIC10491-s1, LIC13050, LIC1 1210, LIC10524, LIC1 1456, LIC12476, LIC1 1570, LIC13244, LIC13238, LIC1 1885, LIC1 1008, LIC13242, LIC1 1336, LIC20250, LIC10525, LIC10464-s2.1, LIC20118, Cop LigAU (unique):Repeats A7′-13, CopLigBU(unique):Repeats B7′-12, CopLigB:Repeats 1-16, nt154-173 and antibody reactive fragments thereof.

32. The antigen composition of claim 31, wherein:
(a) the known reactivities are characterized by strength of interaction with an antibody;
(b) the known reactivities are characterized by an activity state of leptospirosis;
(c) the disease parameter is selected from the groups consisting of a previous or current exposure to leptospirosis, an acute leptospirosis infection, a latent leptospirosis infection, a recurrent leptospirosis infection, a leptospirosis carrier state, and an at least partial immunity to infection with leptospirosis;
(d) the at least two of the antigens are present in at least 40% of a population exposed to leptospirosis;
(e) the known relative antibody reactivity comprises an average antibody binding affinity in the upper tertile of binding affinities of antibodies produced in a leptospirosis patient;
(f) an average quantity of antibodies produced in a leptospirosis patient and directed against the at least two antigen is in an upper tertile of the antibodies produced in the leptospirosis patient;
(g) the carrier is a pharmaceutically acceptable carrier;
(h) the antigen composition further comprises an adjuvant;
fi) the vaccine is a therapeutic vaccine;
(j) the antigen composition comprises at least four antigens; or
(k) at least one of the antigens or fragments thereof are at least partially purified.

33-42. (canceled)

43. A vaccine formulation comprising an antigen composition of claim 1.

44. A vaccine formulation comprising an antigen composition of claim 8.

45. A vaccine formulation comprising an antigen composition of claim 31.
